(19) United States
(12) Patent Application Publication
(10) Pub. No.: US 2007/0060740 A1
(43) Pub. Date: Mar. 15, 2007

(54) Protein crystal

(76) Inventors: Mathias Farnegardh, Ekero (SE); Tornas Bonn, Huddinge (SE); Sherry Sun, Shanghai (CN); Jan Ljunggren, Solna (SE); Harri Ahola, Uppsala (SE); Mats Carlquist, Spanga (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/540,612

(22) PCT Filed: Dec. 24, 2003
(86) PCT No.: PCT/IB03/06412

§ 371 Date: Jul. 24, 2006

(30) Foreign Application Priority Data
Dec. 24, 2002 (GB) 0230177.8

(51) Int. Cl. C07K 14/705; G06F 19/00

(52) U.S. Cl. 530/350; 702/19

(57) ABSTRACT

The present invention is in the fields of biotechnology, protein purification and crystallization, x-ray diffraction analysis, three-dimensional computer molecular modelling and rational drug design. The invention is directed to the Liver X receptor and ligands for this receptor, and in particular to crystalline Liver X receptor beta (LXRβ) and to methods of identifying ligands utilizing LXRβ, as well as to compounds, compositions and methods for selecting, making, and using therapeutic or diagnostic agents having LXRβ modulating or binding activity.

### FIELD OF THE INVENTION

The present invention is in the fields of biotechnology, protein purification and crystallization, x-ray diffraction analysis, three-dimensional computer molecular modelling and rational drug design. The invention is directed to the liver X receptor b (LXRβ, NR1H2) and ligands for this receptor, and in particular to crystalline LXRβ and to methods of identifying ligands utilizing LXRβ, as well as to compounds, compositions and methods for selecting, making, and using therapeutic or diagnostic agents having LXRβ modulating or binding activity.

### BACKGROUND OF THE INVENTION

Liver X receptors are members of the superfamily of nuclear receptors. These transcription factors regulate target genes through a complex series of interactions with specific DNA response elements as well as transcriptional coregulators. The binding of ligand has profound effects on these interactions and has the potential to trigger both gene activation and, in some cases, gene silencing. There are about 50 sequence-related nuclear receptors in humans and the family comprises receptors that recognize hormones, both steroidal and non-steroidal, but also receptors responding to metabolic intermediates and to xenobiotics. There are also a number of so-called orphan receptors where the natural ligand is unknown. Some of the receptors show a very specific and high affinity ligand binding, like the thyroid hormone receptors, while others have a substantially lower affinity for their ligands and are also highly promiscuous in terms of ligand selectivity. Like many of the other non-steroid hormone receptors, LXR functions as a heterodimer with the 9-cis-retinoic acid receptor (RXR) to regulate gene expression. Together with PPARs and FXR LXRs represent a subclass of so called permissive RXR heterodimers. In this subclass, the RXR heterodimers can be activated independently by either the RXR ligand, the partner's ligand or synergistically by both.

LXRs consist of two closely related receptor isoforms encoded by separate genes—LXRα (NR1H3) and LXRβ (NR1H2). As expected, the largest sequence differences are located in the N-terminal domain and in the so-called hinge region connecting the DBD and the LBD. LXRα shows tissue restricted expression with the highest mRNA levels detected in the liver and to a lesser extent in the kidney, small intestine, spleen and adrenal gland. In contrast, LXRβ is ubiquitously expressed Both LXR isoforms have been shown to be activated by specific oxysterols that can be formed in vivo. Recently potent, non-steroidal synthetic ligands have been described. T0901317, GW3965 and F3MethylAA all have binding IC50s around 10 nM.

Important insight into LXR biology has been obtained through the study of LXR deficient mice. Both LXRα and LXRβ knockout mice have been described. The LXRα null strain exhibits a striking inability to metabolize and excrete excess cholesterol when challenged with a high-cholesterol diet. The explanation appears to be an inability to up-regulate the rate-limiting enzyme in cholesterol conversion to bile acid, CYP7A, in response to the excess cholesterol. As a consequence, the conversion of cholesterol to bile-acid that would normally occur is blunted and cholesteryl esters deposit in the liver ultimately resulting in liver-failure. In contrast, the LXRβ knockout strain maintains its natural resistance to a high cholesterol diet These important findings not only prove an important function of LXRα in rodent cholesterol metabolism, but also suggest that the LXR dependent regulation of CYP7A is LXR-subtype selective. The CYP7A LXR response element is not well conserved between rodents and man. LXRs are therefore not expected to be main regulators of cholesterol conversion to bile-acids in humans. This notion is supported by results from in vitro assays using cultured human cells. However, more recently, LXRs have been shown to regulate also several other genes involved in cholesterol and lipid homeostasis. Prominent examples are the phospholipid/cholesteryl ester transporter ABCA1, ABCG1 and the SREBP1c gene that, in turn, induces fatty acid synthesizing enzymes. Increasing insight into the involvement of LXRs in cholesterol and fatty acid homeostasis has led to considerable interest in LXRs as targets for drug development. As an example, one hallmark of atherosclerosis is the build-up of cholesteryl esters in macrophages of the arterial wall, transforming the cells into so-called foam cells that, in turn are constituents of the atherosclerotic plaque. The potential to increase cholesterol efflux from macrophages/foam cells by inducing genes such as ABCA1 and/or G1 thereby preventing or even reversing the atherosclerotic process make LXRs highly interesting drug targets.

The inventor's understanding of how nuclear receptor ligands exert their effects has been dramatically enhanced by the elucidation of the crystal structures of the apo or liganded LBDs of several nuclear receptors. These structures have revealed a common, mainly a helical, fold unique for LBDs of nuclear receptors. It comprises a core layer of three helices (H5/6, H9 and H110) sandwiched between two additional layers of helices (H1′-4 and H7, H8, H11 respectively). This arrangement creates a wedge shaped molecular scaffold that contains a wider upper part, which shows the highest degree of sequence conservation a between the LBDs. The narrower lower part is folded to form a hydrophobic cavity into which the ligand can bind. The remaining secondary elements, an antiparallel b-sheet comprising 2-4 strands and H12 (sometimes also referred to as the AF-2 domain) sits on each side of the ligand-binding cavity. The structures have revealed that ligands can affect the position of H12 so that an agonist puts H12 in a position allowing coactivator binding and preventing corepressor binding, while in an unliganded or antagonist bound receptor the coactivator binding site is blocked. Alternatively, the unliganded or antagonist bound receptor recruits corepressors. The binding modes of several of these coregulators have also recently been depicted in detail.

The present inventors have been able to produce LXRβ crystals and to determine from that the three dimensional structure of the LXRβ ligand binding domain (LBD).

### SUMMARY OF THE INVENTION

The present invention refers to the crystallization of LXRβ and determination of its crystallographic co-ordinates. Therefore, in a first aspect the present invention provides a LXRβ ligand binding domain crystal.

In another aspect of the invention, methods for designing ligands which will bind to LXRβ are provided. Such methods use three-dimensional models based on the crystals of the LXRb ligand-binding domain. Generally, such methods comprise, determining compounds which are likely to bind to the receptor based on their three dimensional shape in particular the ligand binding domain of the LXRb. Preferably, such compounds have a structure that is complementary to the ligand-binding cavity of the LXRb. Such methods comprise the steps of determining which amino acid or amino acids of the ligand-binding domain of the LXRβ interacts with the binding ligand, and selecting compounds or modifying existing compounds, to improve the interaction. Preferably, improvements in the interaction are manifested as increases in the binding affinity but may also include increases in receptor selectivity and/or modulation of efficacy.

Preferably, the ligands bind to the internal LXRβ binding cavity with a high binding affinity, for example within the range of 0.01-1000 nM.

The ligands may bind tightly to the LXRβ yet not up-regulate gene expression thereby inhibiting the action of endogenous LXRβ activators. Thus, the invention also provides a method of inhibiting the activity of endogenous LXRβ activators by providing ligands that bind to LXRβ with a high affinity, blocking the activity of the endogenous ligands. Alternatively, binding of the ligand to the LXRβ may cause conformational changes to the LXRβ inhibiting further binding thereto. The invention further provides a method of inhibiting the activity of endogenous LXRβ ligands in an animal, the method comprising administering to the animal a ligand which binds to at least the LBD, of the LXRβ with high affinity and blocks binding of further ligands to at least the LBD of the LXRβ. Such ligands are potentially useful in, for example, the treatment of LXRβ mediated diseases in humans. Preferably the ligands are identified by the method of designing ligands according to the invention.

### DETAILED DESCRIPTION OF THE INVENTION

One aspect of the invention provides a crystal comprising at least 150 amino acid residues of the LXRβ ligand-binding domain. Preferably, the said crystal comprises at least 200 amino acid residues of LXRβ. More preferably, said crystal contains at least 250 amino acid residues of LXRβ. Most preferably, the said crystal comprises the entire LXRβ amino acid sequence.

Preferably the crystal comprises the amino acid sequence shown as Leu-220 to Asp-458 most preferably Leu-220 to Glu-461 of a LXRβ ligand binding domain as shown in FIG. 5 or an amino acid sequence having at least 95%, especially above 97, 98 or 99% identity to the sequence. This numbering is based on the full sequence of human LXRβ. Preferably, the crystal comprises the entire amino acid sequence shown in FIG. 5.

Isolated protein consisting of the amino acid sequence listed for the crystals are also provided by the invention. The isolated protein may be used to produce the crystals.

The proposed structural identity (based on analogy to the estrogen receptor and thyroid hormone receptor) of parts of the LXRβ ligand-binding domain is shown below, based on the amino acid numbering of the full LXRβ.

| Secondary motif | LXRβ residues |
|---|---|
| Helix-1 | Thr-221 to Val-249 |
| Helix-3 | Ala-261 to Val-289 |
| Helix-4 | Gly-291 to Gln-294 |
| Helix 5 | Gly-296 to Thr-308 |
| Helix 6 | Thr-308 to Arg-319 |
| Sheet-1 | Tyr-320 to His-322 |
| Sheet-2 | Glu-325 to Phe-329 |
| Sheet-3 | Phe-333 to Ser-336 |
| Helix-7 | Ser-336 to Ala-343 |
| Helix-8 | Gln-346 to Gly-364 |
| Helix-9 | Asp-366 to Ser-380 |
| Helix-10 | Pro-389 to Ile-409 |
| Helix-11 | Asp-414 to Gln-445 |
| Helix-12 | Pro-450 to Ile-456 |

An embodiment of this aspect of the invention provides a crystal produced using a sequence including helix 12 of LXRβ. Preferably this is between Pro450 to Ile-456.

The crystals according to the invention may be usable in X-ray crystallography.

In another embodiment of the present invention there is provided a LXRβ crystal as described above also including a ligand bound to LXRβ or a portion thereof. Said ligand may be selected from T0901317 (N-(2,2,2-trifluoroethyl)-N-[4-[2,2,2-trifluoro-1-hydroxy-1-(trifluoromethyl)ethyl]phenyl]-benzenesulfonamide, CAS # [293754-55-9]; WO 00/54759), G-W-3965 (3-(3-(2-chloro-3-trifluoromethylbenzyl-2,2-diphenylethylamino)propoxy)phenylacetic acid, CAS # [405911-09-3]; Collins, Jon L.; et al. J. Med. Chem. (2002), 45(10), 1963-1966), 24(S),25-epoxycholesterol (CAS # [77058-74-3]), N-[1-(2-furanyl)ethyl]-N-4-pyridinyl-tricyclo[3.3.1.13,7]decane-1-carboxamide (CAS # [355833-66-8], WO-01/60818) or any other ligand that binds with reasonably affinity (<1000 nM) to the internal LXRβ binding cavity. The T0901317, G-W-3965 or any other ligand may be used with a coactivator ligand such as TIF2 NR-box 1. 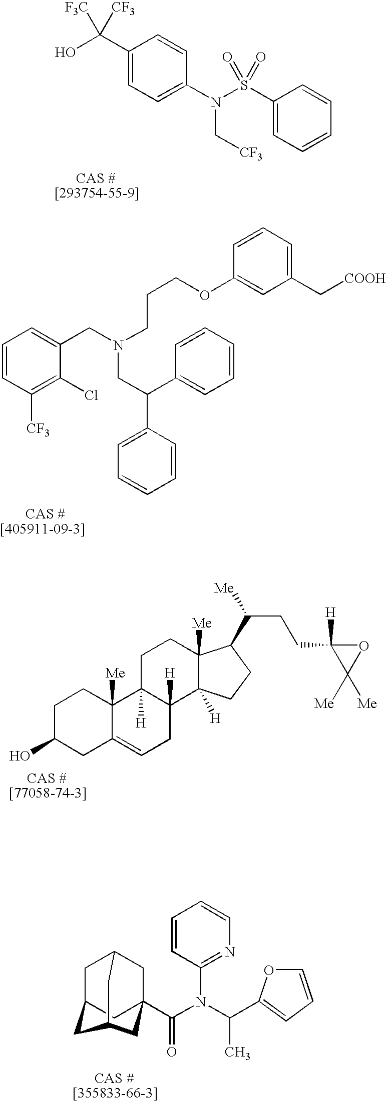 CAS# CAS# CAS# CASE [293754-55-9] [405911-09-3] [77058-74-3] [355833-86-8]

In another embodiment of the present invention there is provided a crystal of LXRβ LBD belonging to the space group P2₁2₁2, and having the unit cell dimensions a=59+/−3 Å, b=100+/−5 Å, c=176+/−3 Å, a=b=g=90°.

In another embodiment of the present invention there is provided a crystal of LXRβ LBD belonging to the space group P6₁22 and having the unit cell dimensions a=59+/−3 Å, b=59+/−3 Å c=294+/−3 Å, a=b=90°, g=120°.

In another embodiment of the present invention there is provided a crystal of LXRβ LDB in complex with a coactivator peptide (such as a peptide corresponding to the first NR-box of TIF2 (Leers, Treuter et al 1998)) belonging to the space group P2₁2₁2 and having the unit cell dimensions a—89+/−3, b=91+/−3, c=131+/−3, a=b=g=90°.

The crystals according to the invention may have a resolution as determined by X-ray crystallography of less than 3.6 Å, preferably less than 2.9 Å.

In another aspect of the present invention, there is provided a machine-readable data storage medium, comprising a data storage material encoded with machine readable data which, when using a machine programmed with instructions for using said data, is capable of displaying a graphical three-dimensional representation of a crystal structure as described above or a homologue of said crystal structure. Homologues include crystals with the same space group, but with another ligand, crystals with the same space group and substantially the same dimensions, and crystals using LXRβ from other species.

In yet another aspect of the present invention, there is provided a method for designing a potential LXRβ ligand for the treatment of diseases modulated by the LXRβ, the method comprising the steps of:
(a) employing computational means to perform a fitting operation between the chemical entity and a binding site of LXRβ identified from a machine-readable storage medium as described above; and
(b) analyzing the results of the fitting operation to predict the association between the potential chemical entity and the binding site.

Preferably the method also comprises the steps of:
(c) synthesizing the potential LXRβ ligand based on the crystal structure of the LXRβ; and
(d) assaying the LXRβ ligand for LXRβ binding, response in a LXRβ reporter cell line, measuring one or more in vivo effects including but not limited to lesion area of fatty streaks in the aortic root, lipoprotein profile and serum triglyceride levels.

The method may alternatively provide the steps of:
synthesising the potential LXRβ ligand based on the crystal structure of said receptor; and
assaying the LXRβ ligand binding response in a LXRβ reporter cell line by measuring one or more in vitro effects, including but not limited to changes in the activity of a LXR response element driven reporter gene such as alkaline phosphatase, green fluorescent protein, or luciferase, changes indicating that the LXRβ ligand may be used for treatment of diseases modulated by LXRβ.

The LXR response element may be provided within, for example, a suitable plasmid containing the response element, reporter gene and suitable termination sequences. The reporter gene will be arranged so that expression of it is under the control of the response element.

Suitable vectors include, but are not limited to, bacterial or eukaryotic vectors such as plasmids or cosmids, phage vectors such as lambda phage, viral vectors such as adenoviral vectors or baculoviral vectors, and other vectors known in the art.

The vector preferably comprises suitable regulatory sequences to allow the nucleic acid molecule of the invention to be expressed in a suitable host cell to produce protein encoded by the nucleic acid molecule. Typically, the vector comprises a suitable promoter and terminator sequences, or other sequences such as poly A sequences, operably linked to the nucleic acid molecule. Such regulatory sequences are well known in the art.

The vector may also comprise a gene to allow the vector to be selected within a cell, such as an antibiotic resistance gene or a nutritional gene. Such genes are well known in the art.

The reporter gene is preferably Green Fluorescent Protein (GFP), which is known in the art. This fluoresces and enables the position of the kinase to be identified.

A further reporter system which may be used is lacZ gene from E. coli. This encodes the β-galactosidase enzyme. This catalyses the hydrolysis of b-galactoside sugars such as lactose. The enzymatic activity in cell extracts can be assayed with various specialised substrates, for example X-gal, which allow enzyme activity quantitation using a spectrophotometer, fluorometer or a luminometer.

Alternatively, the reporter gene may be secreted alkaline phosphatase. This is a secreted enzyme which may be assayed from a supernatent by methods known in the art.

Luciferase, another known reporter gene, may be used. This is derived from the firefly (Photinus pyralis). It catalyses a reaction using D-luciferin and ATP in the presence of oxygen and Mg²⁺ to produce light emission. The amount of light produced, and hence the amount of reporter gene produced under the control of the reporter element, may then be quantified.

The inventors have also identified that helix-12 of LXRβ plays a key role in determining the efficacy (agonism v. antagonism) of a ligand.

Accordingly, preferably the method includes the step of modifying the potential LXRβ ligand so that it:
(a) sterically displaces helix-12; or
(b) disrupts the dimerisation surface.

The dimerisation interface has been identified as helices H10 and H11.

In yet another aspect of the present invention, there is provided a method of designing a ligand which will bind to LXRβ comprising comparing the shape of a compound with the shape of the ligand binding cavity of LXRβ as obtained from a crystal according to the invention, and determining which amino acid or amino acids of the ligand binding domain interact with said compound.

In yet another aspect of the present invention, there is provided a crystallized molecule or molecular complex comprising a binding pocket defined by the structure coordinates of human LXRβ ligand binding domain amino acid residues 200 or a homologue of said molecule or molecular complex wherein said homologue has a root mean square deviation form the backbone atoms of said amino acids of not more than 1.5 Å.

In a preferred embodiment of this aspect there is provided a crystallized molecule or molecular complex comprising a binding pocket defined by the structure coordinates of human LXRβ ligand binding domain amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457 or a homologue of said molecule or molecular complex wherein said homologue has a root mean square deviation form the backbone atoms of said amino acids of not more than 1.5 Å.

A further aspect of the invention provides crystallisable compositions comprising at least 250 amino acid residues of the LXRβ ligand-binding domain.

A further aspect of the invention provides a method of using the crystal of the invention in a drug screening assay comprising:
(a) selecting a potential ligand by performing rational drug design with the three-dimensional structure determined for the crystal, wherein said selecting is performed in conjunction with computer modelling;
(b) contacting (i.e. docking) the potential ligand with the ligand binding domain of LXRβ; and
(c) detecting the binding of potential ligand for the ligand binding domain Preferably, a potential drug is selected on the basis of it having a greater affinity for the ligand domain of LXRβ than that of a standard ligand for the ligand binding domain of LXRβ. Alternatively, potential drugs may be selected by looking for those from a number of potential drugs with the greatest binding affinity.

Preferably the standard ligand in step (c) is T0901317, GW3965, or 24(S),25-epoxycholesterol.

The method may further comprise:
(d) growing a supplemental crystal containing a protein ligand complex formed between the N-terminal truncated LXRβ and the potential drug, wherein the crystal effectively diffracts X-rays for the determination of the atomic coordinates of the protein-ligand complex to a resolution of greater than 5.0 Å;
(e) determining the three-dimensional structure of the supplemental crystal with molecular replacement analysis;
(f) selecting a candidate drug by performing a rational drug design with the three-dimensional structure determined for the supplemental crystal, wherein said selecting is performed in conjunction with computer modelling;
(g) contacting a cell that expresses LXRβ; and
(h) detecting a measure of protein synthesis in the cell; wherein a candidate drug is identified as a drug when it inhibits or enhances the expression of protein synthesis in the cell.

The method preferably comprises an initial step that precedes steps (a) wherein initial step consists of determining the three-dimensional structure of a crystal comprising a protein-ligand complex formed between an N-terminal truncated LXRβ and T0901317, GW3965, or 24(S),25-epoxycholesterol, wherein the crystal effectively diffracts X-rays for the determination of the atomic coordinates of the protein-ligand complex to a resolution of greater than 5.0 Å.

The invention also provides a method of using a crystal of the invention in a drug screening assay comprising:
(a) selecting a potential ligand by performing rational drug design with the three-dimensional structure determined for the crystal, wherein said selecting is performed in conjunction with computer modelling;
(b) adding the potential ligand to a cDNA or protein expression assay regulated by LXRβ;
(c) detecting a measure of a cDNA or protein expression; wherein a potential ligand that regulates the expression of protein expression is selected as a potential drug.

Such cDNA or protein expression assays are themselves known per se in the art. Preferably the assay is in vitro.

Computers for producing a 3D representation are also provided, the representation being of:
(a) a molecule or molecular complex, wherein said molecule or molecular complex comprises a binding pocket defined by the structure coordinates of LXRβ amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457 according to the co-ordinate tables; or
(b) a homolog of said molecule or molecular complex, wherein said homolog comprises a binding pocket that has a root mean square deviation from the backbone atoms of said amino acids of not more than 1.5 Å, wherein said computer comprises:
   (i) a computer-readable data storage medium comprising a data storage material encoded with computer-readable data, wherein said data comprises the structure of LXRβ amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457 according to any one of the co-ordinate tables;
   (ii) a working memory of storing instructions for processing said computer-readable data;
   (iii) a central-processing unit coupled to said working memory and to said computer-readable data storage medium for processing and computer-machine readable data into said three-dimensional representation; and
   (iv) a display coupled to said central-processing unit for displaying said three-dimensional representation.

Preferably the computer produces a 3D representation of:
(a) a molecule or molecular complex defined by structure coordinates of all of the LXRβ ligand binding domain amino acid residues set forth in the co-ordinate tables; or
(b) a homolog of said molecule or molecular complex, wherein said homolog comprises a binding pocket that has a root mean square deviation from the backbone atoms of said amino acids of not more than 1.5 Å; and wherein said computer readable data contains the coordinates of all of the LXRβ ligand binding domain amino acid residues as set forth in any one of the co-ordinate tables.

The invention also provides methods for determining the 3D structure of a complex between LXRβ and a ligand, therefore, which comprises:
(a) obtaining x-ray diffraction data for crystals of the complex; and
(b) utilizing a set of atomic coordinates a portion thereof according to the invention; and coordinates having a root mean square deviation therefrom with respect to conserved protein backbone atoms of not more than 1.5 Å to define the three-dimensional structure of the complex.

A still further aspect of the invention provides a method for determining a modelling structure of a protein containing LXRβ or a complex of said protein and a ligand, which method comprises:
(a) providing a three-dimensional structure defined by a set of coordinates or a portion thereof according to the invention; and coordinates having a root mean square deviation therefrom with respect to conserved protein backbone atoms of not more than 1.5 Å;
(b) generating a three-dimensional model structure of the protein containing LXRβ using a homology modelling method and the structure of step (a) as a template; and
(c) subjecting the resulting model to molecular mechanics energy minimization.

The term “rational drug design”, as used herein, is defined as the designing of drugs for specific purposes, such as the binding to a predetermined receptor or the treatment of a predetermined disease. Examples include the designing of a drug to specifically bind and/or modulate nuclear hormone receptor binding, and the design of drugs to prevent or treat atherosclerosis. This is based upon the knowledge of molecular properties such as binding modes and interaction of the drug to its receptor as revealed by x-ray crystallography; the contribution of various functional groups contained in the drug to the affinity and specificity of the binding of the drug to its target; molecular geometry and electronic structure of drug and its target; and an information catalogued on analogous drug molecules. Such drug design is usually based on computed-assisted modelling and does not usually include pharmacokimetics, dosage analysis or drug administration analysis.

Computer modelling is the theoretical representation of data that simulates the behaviour or activity of systems, processes or phenomena. This includes the use of mathematical equations, computers and other electrical equipment. In the context of drug design, computer modelling allows the simulation of the strength of interaction between a drug conclictal and its target receptor.

Isolated proteins consisting essentially of the LBD of LXRβ, vectors encoding such proteins and host cells are also provided. the isolated protein may be attached to a tag, such as a his-tag.

Drug candidates are potential drugs. That is, they include compounds which have initial indications that they will have potential clinical use or activity.

The term “supplemental crystal” refers to a second, additional, crystal complexed with a further, different LXRβ ligand.

The term “standard ligand” refers to a known, characterised, ligand.

### Structure Based Design of LXR Ligands

The present invention elucidates the structure of the ligand-binding cavity of LXRβ.

Knowledge of the structure of this cavity has utility in the design of structurally novel LXRβ ligands and in the design of non-obvious analogues of known LXRβ ligands with improved properties. These enhanced properties include one or more of the following: (1) higher affinity, (2) improved selectivity for LXRβ vs. related nuclear hormone receptors and/or (3) a designed degree of efficacy (agonism vs. partial agonism vs. antagonism). Without knowledge of the LXRβ structure, modifications to produce ligands with enhanced properties and a reasonable likelihood of success would not be available to those skilled in the art. The LXRβ structure also has utility in the discovery of new, structurally novel classes of LXRβ ligands. Electronic screening of large, structurally diverse compound libraries such as the Available Chemical Directory (ACD) will identify new structural classes of LXRβ ligands which will bind to the 3-dimensional structure of the LXRβ. Additionally the LXRβ structure allows for “reverse-engineering” or “de novo design” of compounds to bind to LXRβ.

(1) Enhanced Affinity

The present invention has revealed the size and shape of the interior binding cavity for representative LXRβ ligands T0901317 and GW-3965. The sizes and shapes of the cavities were delineated using the PASS program (“Fast Prediction and Visualization of Protein Binding Pockets With PASS”; G. P. Brady, Jr. and P. F. W. Stouten; J. Comp.-Aided Mol. Design, 14: 383-401, 2000). The interior binding cavity of LXRβ/T0901317 complex is shown in FIG. 6 (left) and has the dimensions of 13.1×9.2×7.5 Å along the first, second, and third principle moments of inertia respectively. The interior binding cavity of LXRβ/GW-3965 complex is shown in FIG. 6 (right) and has the dimensions of 17.0×11.9×8.0 Å along the first, second, and third principle moments of inertia respectively. In addition, this structure reveals a narrow “water-channel” adjacent to the cavity occupied by T0901317 and GW-3965.

Ligands which occupy as much of the interior binding cavities including the unoccupied “water-channels” as revealed by the LXRβ/T0901317 and LXRβ/GW-3965 complexes without sterically colliding with the receptor will provide ligands with higher affinity than either T0901317 or GW-3965.

The present invention has also revealed the presence of a histidine residue (His-435) which forms a very strong hydrogen bond with the acidic hydroxyl group of the ligand TO901317 [Ne−OC(CF₃)₂Ar) distance=2.6 Å]. In addition, the sulfonyl oxygen atom of ligand TO901317 forms a weak hydrogen bond to the Ser-278 (Og−O═S═O distance=4.1 Å). New ligands which preserve the strong hydrogen bond by an appropriately placed acidic hydrogen atom to interact with the Ne atom of His-435 and in addition place a hydrogen bond donating group closer to the Og atom of Ser-278 will show enhanced affinity for LXRβ relative to TO901317.

The present invention also reveals that there are a number of unsatisfied hydrogen bond partners in the ligand binding cavity (see FIG. 7). These include the backbone carbonyl group of Phe-271 and the sidechain Og atoms of Thr-272 and Thr-316. Introduction of appropriately positioned hydrogen bond donating substituents on the ligand which form strong hydrogen bonds to one or more of these three hydrogen bond accepting groups in the receptor binding cavity will serve to enhance affinity.

The ligands produced in accordance with the invention bind more effectively to the LXRβ than TO901317. The ligand may bind with twice the binding affinity of TO901317, preferably three times the affinity, and most preferably ten or more times the affinity.

Preferably, the ligand produced in accordance with the invention occupies as much of the interior binding cavities of LXRβ as revealed by the LXRβ/TO901317 and LXRβ/GW-3965 complexes without perturbing the remainder of the LXRβ structure.

Preferably, the ligand produced in accordance with the invention also forms a hydrogen bond with the Ne atom of His-435 and at least one additional hydrogen bond to either Phe-271 (backbone carbonyl group), Thr-272 (Og), Ser-278 (Og), or Thr-316 (Og) of LXRβ without perturbing the remainder of the LXRβ structure.

(2) Improved Selectivity

The LXRβ receptor is very closely related to the LXRα and relatively closely related to the RXR, PXR, FXR, PPAR receptors. The RXR, PXR, FXR, PPAR receptors differ significantly in their primary sequence and slightly in their tertiary structure. As a consequence of these receptor differences, ligands may bind with different affinity to these four receptors.

The closest amino acid difference between LXRα and LXRβ in the vicinity of the bound ligand is Ala-294(a)/Thr-308(b). This is in turn next to Met-298(a)/312(b) which directly lines the binding cavity. Rotation about the c₃ sidechain of to Met-298(a) is more facile in LXRα than in LXRβ due to the presence of the smaller Ala-294(a) residue. Therefore subsituents from the ligand which push on Met-298(a) will afford ligand that are selective for LXRα over LXRβ.

Furthermore, a detailed understanding of the different receptors enables the different behaviour of a compound in different tissues to be understood, for example the selective liver X receptor modulators (SLXRMs) on the tissue in which it is active. LXRα and LXRβ have different tissue distributions and therefore ligands which display LXR isoform binding selectivity will also display tissue selectivity.

The present invention provides new ligands which exploit these differences by positioning ligand substituents in close proximity to one or more amino acid residue that differ between LXRβ and RXR, PXR, FXR, PPAR.

The ligands produced in accordance with the invention bind more effectively to the LXRβ receptor than to the RXR, PXR, FXR, or PPAR receptor. The selectivity of the binding to the LXRβ receptor may be tenfold, more preferably one hundred-fold, and most preferably greater than one thousand-fold.

(3) Modulation of Efficacy

This invention provides an understanding of the differences between LXRβ agonist and antagonist binding and therefore a means to design LXRβ ligands with the desired degree of efficacy. An examination of the differences between the ERa/estradiol (agonist; PDB accession code: 1ERE) and ERb/raloxifene (agonist; PDB accession code: 1ERR) complexes reveals a large movement in Helix-12. H12 adopts an “agonistic” conformation defined by the structure of the ERa/estradiol complex and an “antagonistic” conformation defined by the structure of the ERb/raloxifene complex. These two conformations are in thermodynamic equilibrium. When the ER is complexed with a full agonist, such as estradiol, the equilibrium lies far in the direction of the “agonistic” conformation. In contrast, while when complexed with an antagonist, the equilibrium is pushed in the direction of the “antagonistic” conformation. In the case of raloxifene ER ligand, the bulky side-chain collides with H12 in its agonistic conformation, thereby driving the equilibrium in the antagonistic direction. By introduction of progressively shorter side chains in raloxifene, the equilibrium will be gradually shifted back towards the agonist conformation. By analogy, replacement of one of the fluorine atoms of the hexafluoroisopropanol group of TO901317 will sterically collide with H12 in LXRβ. Thus, this invention provides a means of developing ligands with the desired degree of efficacy (agonist, partial agonist, or antagonist).

In particular, the importance of H12 has been determined as playing a central role in determining the efficacy (agonism vs. antagonism) of a ligand. Thus, ligands which are able to bind to and/or alter the conformation of H12 are of particular importance when designing a ligand or assessing the binding of a ligand, for the LXRβ receptor.

Additionally, it has been found that at least the majority of such receptor proteins when activated by binding to an agonist ligand are in the form a dimer (Khorasanizadeh S, Rastinejad F. 2001). Such dimerization leads to a potential route for disruption. Disruptions of this type can be used to predict antagonism or to produce antagonists. Disruptions may take the form of ligand binding which alters the conformation of the helices that comprise the dimerization interface or direct binding to the dimerization interface which then inhibits dimerization.

Further, the orientation of the ligand may be keyed to the receptor, in the dimeric or monomeric form. Furthermore, using the crystals of the present invention, the influence of ligand binding to the LDB on the receptor conformation can now be shown to have influences on the behaviour of the receptor since it may disrupt the binding of co-activator, co-repressor, or heat-shock proteins. Previously, such predictions could not me made.

### Production of Liver X Receptor B Crystals and their Application

The present inventors have been able to isolate, differentiate and produce crystals for the liver X receptor b.

The crystal may be produced from a sequence comprising at least 250 amino acids, and preferably at least 200 amino acids of LXRβ. More preferably, the sequence comprises at least a portion of the ligand-binding domain of LXRβ. Alternatively, the sequence comprises the whole ligand-binding domain of LXRβ.

Advantageously, the crystals have a resolution determined by X-ray crystallography of less than 3.6 Å and most preferably less than 2.9 Å.

The production of such crystals has enabled the three dimensional structure of the ligand binding domain of LXRβ to be mapped. Use of such crystals in conjunction with the map enables a better understanding of how T0901317, GW3965 and other ligands bind to LXRβ with precision. This technique can also enable the design of receptor selective LXRβ agonists and antagonists since now the precise differences in the binding sites between LXRβ and the closely related LXRα.

Crystals of the LXRβ ligand-binding domain can be used as models in methods for the design of synthetic compounds intended to bind to the receptor. Such models show why very slight differences in chemical moieties of a ligand potentially have widely varying binding affinities. Hence, the three dimensional structure of the ligand binding domain can be used as a pharmaceutical model for compounds which bind to Liver X receptors.

Embodiments of the invention will now be described in more detail, by way of example, with reference to the accompanying drawing.

### FIGURE LEGENDS

FIG. 1. Cartoon view of the LXRβ receptor with labeled helices.

FIG. 2 shows representative portions of a 2.4 Å resolution SigmaA weighted 2 Fobs-Fcalc map where Fobs are the observed and Fcalc are the calculated structure-factor amplitutes and 2Fobs-Fcalc is the difference Fourier synthesis electron density map in which model error is reduced and electron density at the chosen contour (mesh diagram) approximates the molecular surface for the LXRβ/GW3965 complex. The structure of GW3965 (tube diagram) is fitted to the experimental electron density (mesh diagram).

FIG. 3. Superposition of the LXRβ/T0901317 (carbons black) and the LXRβ/GW3965 (carbons light grey) complexes reveal dramatic changes in the ligand-binding pocket.

FIG. 4. Residues that are within hydrogen bond distance or van der Waals (4.2 Å) distance to the ligand are labeled. Dashed lines indicate hydrogen bonds and lines indicate Van der Waals interactions. These interactions are shown in (a) for the LXRβ/T0901317 complex, and in (b) for the LXRβ/GW3965.

FIG. 5(a). Full length natural sequence of human LXRβ.

FIG. 5(b). The crystallized protein sequence with the first four non-LXRβ residues gshm and the remaining 213-416 originating from human LXRβ.

FIG. 6. Interior binding cavity of the LXRβ/T0901317 complex (left) and LXRβ/GW-3965 (right). The Ca-trace of the protein is represented by solid line. The structure of the ligand T0901317 and GW-3965 ligands are represented by a ball-and-stick diagram. The binding cavity is represented by a transparent surface which is filled by PASS probe spheres (dots).

FIG. 7. Unsatisfied hydrogen bonding partners (backbone carbonyl groups of Phe-266, Phe-271, Met-312 and side-chain hydroxyl groups of Thr-272, Thr-316) as revealed by the LXRβ/T0901317 complex. Structure of T0901317 is represented by a capped sticks figure surrounded by the interior binding cavity of the receptor (transparent surface). Key amino acid residues are represented by labeled capped-stick. Hydrogen bonding accepting sites on the surface of the receptor binding cavity are represented by solid surfaces.

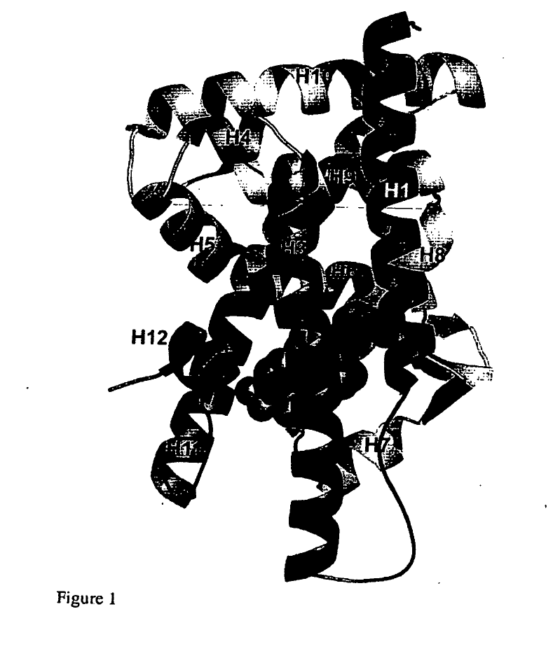

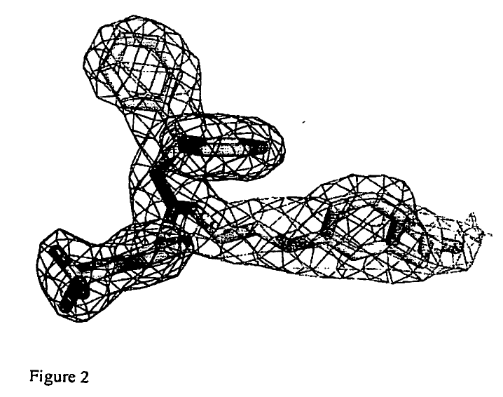

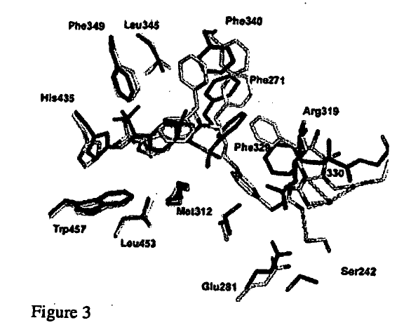

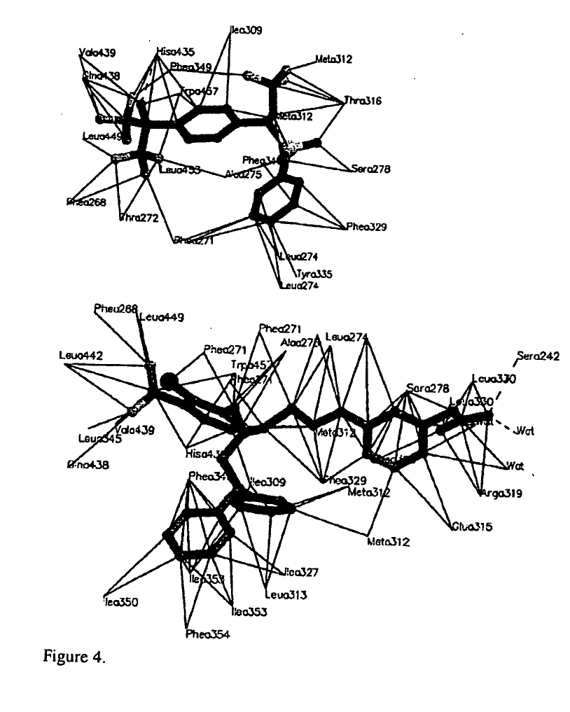

DNA Construction Work

The human LXRβ sequence is publicly available with accession number P55055 (SwissProt.) (Shinar, D. M. et al. (1994)). A construct spanning Gly213-Glu461 with the addition of an N-terminal 6×His tag was used in the present work. The His-tag was designed to be cleavable using thrombin.

Protein Production

The protein was expressed in Escherichia coli BL21 Star™ (DE3) cells (Invitrogen) using the pET28a expression system. Fermentation was carried out in batch culture (2xLB medium, 22° C.) and expression of the recombinant protein was induced by the addition of 0.55 mM IPTG (isopropyl-β-D-thiogalactoside) at OD₆₀₀=5.0. After 4 h of induction the cells were harvested by centrifugation. The cell pellet was resuspended and washed once with buffer (20 mM HEPES pH 8.0, 100 mM KCl, 10% glycerol and 2.5 mM monothioglycerol). Final cell pellet was frozen at −70° C. 40 g cells were lysed by glass beadbeater (BioSpec Products, Inc.) in extract buffer containing 50 mM Tris, pH8.8, 250 mM NaCl, 10% glycerol and 1 mM PMSF. Soluble protein extract were collected by centrifugation at 11000 rpm, 20 min in Sorvall RC-5B centrifuge (Du Pont-instrument AB), GSA rotor.

Protein Purification

Crude LXRβ was eluted from 25 ml Talon by 20 mM Tris, pH8.0, 100 mM imidazole. Further purification was achieved using anion-exchange chromatography (5 ml Hitrap Q FF ion exchange column, Amersham Bioscience), and applying a gradient from 0 to 250 mM NaCl, pH8.0, eluted LXRβ. After thrombin cleavage, the final LXRβ (6-7 mg) fraction was obtained by running 4% acryl amide native gel electrophoresis in Tris-Epps buffer system.

Protein Quality Analysis

To elucidate the homogeneity of LXRβ, throughout the purification samples were collected and run on SDS and native PAGE gels (Phast, Amersham Biosciences, Sweden). Reverse phase HPLC runs were performed on a Waters HPLC system (Waters, USA) at denaturing conditions. Typically, 100 ml sample was acidified by addition of 10% acidic acid (final concentration). A sample was injected and eluted in a 25-75% acetonitrile-water gradient in 0.1% triflouroacidic acid at 1 ml/min. The method proved to be very useful to reveal problems with ligand binding and LXRβ stability and for determine the concentration and LXRβ-ligand ratio.

Crystallization and Data Collection

Crystallization was carried out using the hanging drop vapour-diffusion technique. Both LXRβ-GW9365 and LXRβ-T0901317 crystals were grown from buffer containing 8.5% iso-propanol, 17% PEG 4000, 85 mM HEPES, pH7.5, and 15% Glycerol at room temperature. The first LXRβ/T0901317 crystals formed in the P6122 space group, with a=b=58.7, c=293.8 and diffracted to better than 3 Å. In the same drops another crystal form was later detected belonging to the P212121 space group. Before data collection, crystals were flash-frozen in the 100 K nitrogen gas stream of an Oxford cryostream700. Data was either collected with an MAR345 image plate detector using X-rays from a Rigaku H3R rotating anode generator+Osmic Confocal Max-Fluxa optics or with a ADSC Q4R CCD at Experimental Station ID14-4 at ESRF. The observed reflections where reduced, merged and scaled with MOSFLM, and Scala in the CCP4 package.

Structure Determination and Refinement

The structure was determined by molecular replacement methods with the CCP4 AmoRe program (Acta. Cryst. D50 (1994), pages 760-763), using an LXRβ homology model based on a thyroid hormone receptorb structures (Protein Databank Accession Code INAX). A publicly available structure such as 1bsx.pdb, from the Protein Data Bank, could also have been used to create the model. The molecular replacement was done on the first 3 Å data of LXRβ/T0901317 crystallized in P6122 and revealed one monomer per asymmetric unit. The crystal packing along one of the 2-folds revealed that the protein formed a tight homodimer, which allowed us to use the homodimer to search the second crystal form P212121 that gave 2 homodimers in the asymmetric unit. Electron densities for the T0901317 ligand confirmed the solutions of the molecular replacement. Model building was done with O and refinement initially with CNX and later with the CCP4 Refmac program and manual rebuilding. The four monomer complexes where treated as single TLS groups in Refmac which gave more interpretable electron density maps and improved the R-factors substantially.

TABLE 1
Summary of data collection, processing and refinement.

| Complex | LxRβ/T0901317 | LxRβ/GW3965 |
|---|---|---|
| Data collection | In house | ID14 EH4 ESRF |
| Source | | |
| Space group | P212121 | P212121 |
| Unit cell parameters | | |
| a | 58.7 | 58.7 |
| b | 103.3 | 98.9 |
| c | 176.0 | 175.8 |
| Resolution | 2.8 Å | 2.4 (2.4-2.53) |
| | (2.8-2.95 Å) | |
| Observations | | |
| Unique | 27153 | 37733 |
| Total | 92460 | 1129438 |
| Completeness (%) | 99.9 (99.7) | 98.5 (95.4) |
| <I>/<s(I)> | 7.6 (1.9) | 8.8 (3.5) |
| Rsym % | 8.4 (40.2) | 5.0 (21.8) |
| Refinement | | |
| Rwork | 19.5 (27.9) | 20.7 (21.8) |
| Rfree | 26.2 (34.8) | 26.3 (29.6) |
| Number of atoms | 7782 | 7673 |
| R.m.s deviation | | |
| Bonds (Å) | 0.016 | 0.016 |
| Angles (°) | 1.49 | 1.36 |
| Average B-factor | 24.3 | 23.1 |
| (Å²) | | |

### REFERENCES

Alberti, S., G Schuster, et al. (2001), “Hepatic cholesterol metabolism and resistance to dietary cholesterol in LXRbeta-deficient mice”, J. Clin. Invest., 107(5): 565-73.
Apfel, R., D. Benbrook, et al. (1994), “A novel orphan receptor specific for a subset of thyroid hormone-responsive elements and its interaction with the retinoid/thyroid hormone receptor subfamily”, Molecular & Cellular Biology, 14(10): 7025-35.
Chiang, J. Y., R. Kimmel, et al. (2001), “Regulation of cholesterol 7alpha-hydroxylase gene (CYP7A1) transcription by the liver orphan receptor (LXRalpha)”, Gene, 262(1-2): 257-265.
Collins, J. L., A. M. Fivush, et al. (2002), “Identification of a Nonsteroidal Liver X Receptor Agonist through Parallel Array Synthesis of Tertiary Amines”, J. Med. Chem., 45(10): 1963-6.
Costet, P., Y. Luo, et al. (2000), “Sterol-dependent transactivation of the ABC1 promoter by the liver X receptor/retinoid X receptor”, J. Biol. Chem., 275(36): 28240-5.
Janowski, B. A., M. J. Grogan, et al. (1999), “Structural requirements of ligands for the oxysterol liver X receptors LXRalpha and LXRbeta”, Proceedings of the National Academy of Sciences of the United States of America, 96(1): 266-71.
Janowski, B. A., P. J. Willy, et al. (1996), “An oxysterol signalling pathway mediated by the nuclear receptor LXRalpha”, Nature, 383(6602): 728-31.
Khorasanizadeh, S. and F. Rastinejad (2001), “Nuclear-receptor interactions on DNA-response elements”, Trends Biochem. Sci., 26(6): 384-90.
Leers, J., E. Treuter, et al. (1998), “Mechanistic principles in NR box-dependent interaction between nuclear hormone receptors and the coactivator TIF2”, Mol Cell Biol., 18(10): 6001-13.
Lehmann, J. M., S. A. Kliewer, et al. (1997), “Activation of the nuclear receptor LXR by oxysterols defines a new hormone response pathway”, Journal of Biological Chemistry, 272(6): 3137-40.
Menke, J. G., K. L. Macnaul, et al. (2002), “A novel liver X receptor agonist establishes species differences in the regulation of cholesterol 7alpha-hydroxylase (CYP7a)”, Endocrinology, 143(7): 2548-58.
Peet, D. J. B. A. Janowski, et al. (1998), “The LXRs: a new class of oxysterol receptors. [Review] [37 refs]”, Current Opinion in Genetics & Development, 8(5): 571-5.
Peet, D. J., S. D. Turley, et al. (1998), “Cholesterol and bile acid metabolism are impaired in mice lacking the nuclear oxysterol receptor LXRalpha”, Cell, 93(5): 693-704.
Repa, J. J., G. Liang, et al. (2000), “Regulation of mouse sterol regulatory element-binding protein-1c gene (SREBP-1c) by oxysterol receptors, LXRalpha and LXRbeta”, Genes Dev, 14(22): 2819-2830.
Repa, J. J., S. D. Turley, et al. (2000), “Regulation of absorption and ABC1-mediated efflux of cholesterol by RXR heterodimers [see comments]”, Science, 289(5484): 1524-9.
Schultz, J. R., H. Tu, et al. (2000), “Role of LXRs in control of lipogenesis”, Genes Dev., 14(22): 2831-2838.
Schwartz, K., R. M. Lawn, et al. (2000), “ABC1 gene expression and ApoA-1-mediated cholesterol efflux are regulated by LXR”, Biochemical & Biophysical Research Communications, 274(3): 794-802.
Shinar, D. M., N. Endo, et al, (1994), “NER, a new member of the gene family encoding the human steroid hormone nuclear receptor”, Gene, 147(2): 273-6.
Song, C., J. M. Kokontis, et al. (1994), “Ubiquitous receptor: a receptor that modulates gene activation by retinoic acid and thyroid hormone receptors”, Proceedings of the National Academy of Sciences of the United States of America, 91(23): 10809-13.
Teboul, M. E. Enmark, et al. (1995), “OR-1, a member of the nuclear receptor superfamily that interacts with the 9-cis-retinoic acid receptor”, Proceedings of the National Academy of Sciences of the United States of America, 92(6): 2096-100.
Venkateswaran, A., B. A. Laffitte, et al. (2000), “Control of cellular cholesterol efflux by the nuclear oxysterol receptor LXRalpha”, Proc. Natl. Acad. Sci. USA, 97(22): 12097-12102.
Venkateswaran, A., J. J. Repa, et al. (2000), “Human white/murine ABC8 mRNA levels are highly induced in lipid-loaded macrophages. A transcriptional role for specific oxysterols”, J. Biol. Chem., 275(19): 14700-7.
Willy, P. J. and D. J. Mangelsdorf (1997), “Unique requirements for retinoid-dependent transcriptional activation by the orphan receptor LXR”, Genes & Development, 11(3): 289-98.

Willy, P. J. K. Umesono, et al. (1995), “LXR, a nuclear receptor that defines a distinct retinoid response pathway”, Genes & Development, 9(9): 1033-45.

LENGTHY TABLE REFERENCED HERE
US20070060740A1-20070315-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070060740A1). An electronic copy of the table will also be available from the USPTO upon request and payment of the fee set forth in 37 CFR 1.19(b)(3).

1. A crystal comprising at least 150 amino acid residues of the LXRβ ligand binding domain.

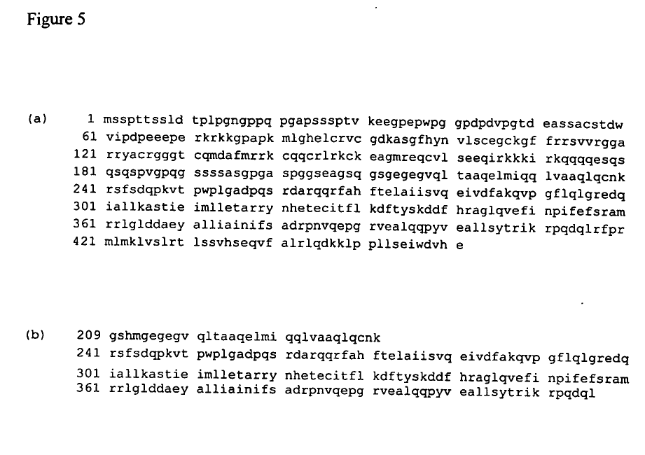

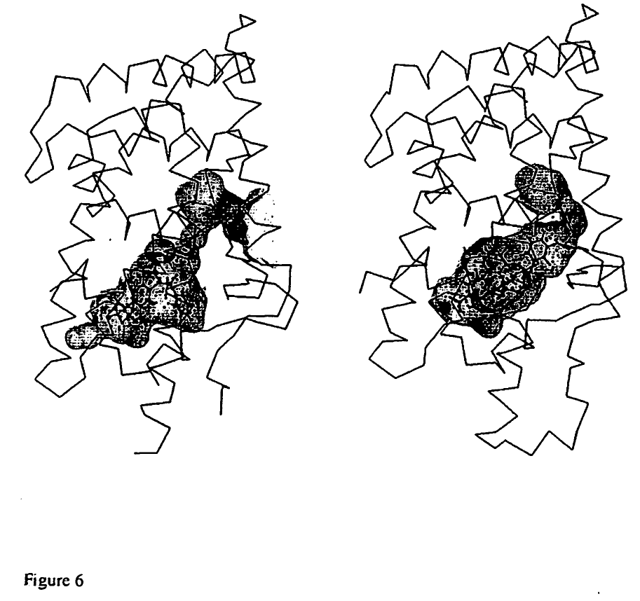

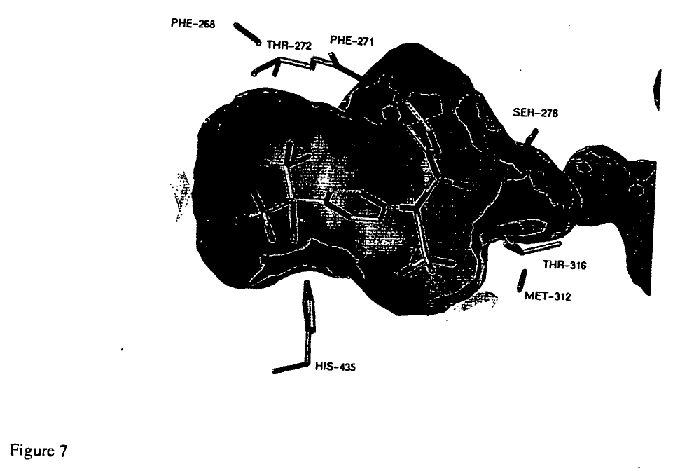

2. A crystal according to claim 1 comprising the amino acid sequence from Leu-220 to Glu-461 of a human LXRβ shown in FIG. 5a (SEQ ID NO: 1) or an amino acid sequence having at least 95% identity with the sequence and which encodes for a LXRβ ligand binding domain.

3. A crystal according to claim 1, comprising the entire LXRβ ligand binding domain.

4. A crystal according to claim 1, produced using a sequence including helix 12 of LXRβ.

5. A crystal according to claim 1, usable in X-ray crystallography.

6. A crystal according to claim 1, including a ligand bound to LXRβ or a portion thereof.

7. A crystal according to claim 6 in which the ligand is T0901317, GW3965 or any other ligand that binds with an affinity of IC₅₀<1000 nM to the internal LXRβ binding cavity.

8. A crystal of LXRβ LBD belonging to the space group P2₁2₁2, and having the unit cell dimensions a=59+/−3 Å, b=100+/−5 Å, c=176+/−3 Å, α=β=γ=90°.

9. A crystal of LXRβ LBD belonging to the space group P6₁22 and having the unit cell dimensions a=59+/−3 Å b=59+/−3 Å c=294+/−3 Å, α=β=90°, γ=120°.

10. A crystal of LXRβ LBD in complex with a coactivator peptide (TIF2 NR-box 1) belonging to the space group P2₁2₁2 and having the unit cell dimensions a=89+/−3, b=91+/−3, c=131+/−3, α=β=γ=90°.

11. A crystal according to claim 1, having a resolution determined by X-ray crystallography of better than 3.6 Å.

12. A crystal according to claim 11 having a resolution determined by X-ray crystallography of better than 2.9 Å.

13. A drug screening assay comprising the steps of:
(a) selecting a potential ligand by performing rational drug design with the three-dimensional structure determined for the crystal of claim 1, wherein said selecting is performed in conjunction with computer modelling;
(b) contacting (i.e. docking) the potential ligand with the ligand binding domain of LXRβ; and
(c) detecting the binding of the potential ligand for the ligand binding domain.

14. A method according to claim 13, wherein a potential drug is selected on the basis of it having a greater affinity for the ligand domain of LXRβ than that of a standard ligand for the ligand binding domain of LXRβ.

15. The method of claim 14 wherein the standard ligand in step (c) is T0901317, GW3965, or 24(S),25-epoxycholesterol.

16. The method of claim 13, further comprising the step of:
(d) growing a supplemental crystal containing a protein ligand complex formed between the N-terminal truncated LXRβ and the potential drug, wherein the crystal effectively diffracts X-rays for the determination of the atomic coordinates of the protein-ligand complex to a resolution of greater than 5.0 Å;
(e) determining the three-dimensional structure of the supplemental crystal with molecular replacement analysis;
(f) selecting a candidate drug by performing a rational drug design with the three-dimensional structure determined for the supplemental crystal, wherein said selecting is performed in conjunction with computer modelling;
(g) contacting a cell that expresses LXRβ; and
(h) detecting a measure of protein synthesis in the cell; wherein a candidate drug is identified as such a drug when it inhibits or enhances the expression of protein synthesis in the cell.

17. The method of claim 16 further comprising an initial step that precedes steps (a) wherein initial step consists of determining the three-dimensional structure of a crystal comprising a protein-ligand complex formed between an N-terminal truncated LXRβ and T0901317, GW3965, or 24(S),25-epoxycholesterol, wherein the crystal effectively diffracts X-rays for the determination of the atomic coordinates of the protein-ligand complex to a resolution of greater than 5.0 Å.

18. A drug screening assay comprising the steps of:
(a) selecting a potential ligand by performing rational drug design with the three-dimensional structure determined for the crystal of claim 1, wherein said selecting is performed in conjunction with computer modelling;
(b) adding the potential ligand to a cDNA or protein expression assay regulated by LXRβ; and
(c) detecting a measure of a cDNA or protein expression; wherein a potential ligand that regulates the expression of protein expression is selected as a potential drug.

19. The method of claim 18 wherein said protein expression is an in vitro protein expression assay.

20. A machine-readable data storage medium, comprising a data storage material encoded with machine readable data which, when using a machine programmed with instructions for using said data, is capable of displaying a graphical three-dimensional representation of a crystal structure according to claim 1 or a homologue of said crystal structure.

21. A method for designing a potential LXRβ ligand for the treatment of diseases modulated by the natural LXRβ ligand, the method comprising the steps of:
(a) performing a fitting operation between the chemical entity and a binding site of LXRβ receptors identified from a machine-readable storage medium according to claim 20; and
(b) analyzing the results of the fitting operation to predict the association between the potential LXRβ ligand and the binding site.

22. Method according to claim 21, additionally providing the steps of:
(c) synthesizing the potential LXRβ ligand based on the crystal structure of the said receptor; and
(d) assaying the LXRβ ligand binding response in a LXRβ animal model cell line by measuring one or more in vivo effects including but not limited to changes in lipoprotein profile, changes in serum or tissue triglyceride levels, changes in serum or tissue cholesterol levels, changes in serum glucose levels, changes in atherosclerotic lesion size indicating that the LXRβ ligand may be used for treatment of diseases modulated by LXRβ.

23. A method according to claim 21, additionally providing the steps of:
(e) synthesising the potential LXRβ ligand based on the crystal structure of said receptor; and
(f) assaying the LXRβ ligand binding response in a LXRβ reporter cell line by measuring one or more in vitro effects, including but not limited to changes in the activity of a LXR response element driven reporter gene such as alkaline phosphatase, green fluorescent protein, or luciferase, changes indicating that the LXRβ ligand may be used for treatment of diseases modulated by LXRβ.

24. A method according to claim 21, additionally comprising the steps of modifying the potential LXRβ ligand so that it:
(a) sterically displaces helix-12; or
(b) disrupts the dimerisation surface.

25. A method according to claim 21, wherein said a potential LXRβ ligand is a LXRβ antagonist.

26. A method according to claim 21, wherein said potential LXRβ ligand is an agonist.

27. A method according to claim 21, wherein said potential LXRβ ligand is a selective modulator.

28. A method of designing a ligand which will bind to LXRβ comprising comparing the shape of a compound with the shape of the ligand-binding cavity of LXRβ as obtained from a crystal according to claim 1, and determining which amino acid or amino acids of the ligand binding domain interact with said compound.

29. A crystallized molecule or molecular complex comprising a binding pocket defined by the structure coordinates of human LXRβ ligand binding domain amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457, according to the co-ordinate tables or a homologue of said molecule or molecular complex wherein said homologue has a root mean square deviation form the backbone atoms of said amino acids of not more than 1.5 Å.

30. A crystallisable composition comprising at least 150 amino acid residues of the LXRβ ligand-binding domain.

31. An isolated protein consisting essentially of the amino acid sequence shown from amino acid 220 to amino acid 461 in FIG. 5a (SEQ ID NO: 1) or the sequence shown in FIG. 5b (SEQ ID NO: 2).

32. An isolated protein according to claim 31, additionally comprising a tag, such as a his-tag.

33. A vector, such as a plasmid, containing a nucleic acid molecule encoding a protein consisting of the amino acid sequence shown from 220 to 461 in FIG. 5a (SEQ ID NO: 1) or the sequence shown in FIG. 5b (SEQ ID NO: 2).

34. A host cell containing a vector according to claim 33.

35. An isolated protein having an amino acid sequence identical to the amino acid sequence used in a crystal according to claim 1.

36. A computer for producing a three-dimensional representation of:
(a) a molecule or molecular complex, wherein said molecule or molecular complex comprises a binding pocket defined by the structure coordinates of LXRβ amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457 according to the co-ordinate tables; or
(b) a homolog of said molecule or molecular complex, wherein said homolog comprises a binding pocket that has a root mean square deviation from the backbone atoms of said amino acids of not more than 1.5 Å, wherein said computer comprises:
(i) a computer-readable data storage medium comprising a data storage material encoded with computer-readable data, wherein said data comprises the structure of LXRβ amino acid residues Ser242, Phe268, Phe271, Thr272, Leu274, Ala275, Ser278, Ile309, Met312, Leu313, Glu315, Thr316, Arg319, Ile327, Phe329, Leu330, Tyr335, Phe340, Leu345, Phe349, Ile350, Ile353, Phe354, His435, Gln438, Val439, Leu442, Leu449, Leu453, Trp457 according to the co-ordinate tables;
(ii) a working memory of storing instructions for processing said computer-readable data;
(iii) a central-processing unit coupled to said working memory and to said computer-readable data storage medium for processing and computer-machine readable data into said three-dimensional representation; and
(iv) a display coupled to said central-processing unit for displaying said three-dimensional representation.

37. The computer according to claim 36 wherein said computer produces a three-dimensional representation of:
(a) a molecule or molecular complex defined by structure coordinates of all of the LXRβ ligand binding domain amino acid residues set forth in the co-ordinate tables; or
(b) a homolog of said molecule or molecular complex, wherein said homolog comprises a binding pocket that has a root mean square deviation from the backbone atoms of said amino acids of not more than 1.5 Å; and wherein said computer readable data contains the coordinates of all of the LXRβ ligand binding domain amino acid residues as set forth in the co-ordinate tables.

38. A method for determining the three-dimensional structure of a complex between LXRβ and a ligand therefore, which comprises:
(a) obtaining x-ray diffraction data for crystals of the complex as defined in claim 1; and
(b) utilizing a set of atomic coordinates as defined in claim 29 or a portion thereof; and coordinates having a root mean square deviation therefrom with respect to conserved protein backbone atoms of not more than 1.5 Å to define the three-dimensional structure of the complex.

39. A method for determining a modelling structure of a protein containing LXRβ or a complex of said protein and a ligand, which method comprises:
(a) providing a three-dimensional structure defined by a set of coordinates as defined in claim 29, or a portion thereof; and coordinates having a root mean square deviation therefrom with respect to conserved protein backbone atoms of not more than 1.5 Å;
(b) generating a three-dimensional model structure of the protein containing LXRβ using a homology modelling method and the structure of step (a) as a template; and
(c) subjecting the resulting model to molecular mechanics energy minimization.


SEQUENCE LISTING (2 sequences)

SEQ ID NO: 1 - protein, 461 aa, Homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 2 - protein, 208 aa, Artificial Sequence
  The crytallised protein sequence with the first four non-LXR Beta amino acid residues (GSHM) fused to the N-terminal end of residues 213-416 originating from human LXR Beta
GSHMGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQL